(19) United States
(12) Patent Application Publication
(10) Pub. No.: US 2026/0315339 A1
(43) Pub. Date: Oct. 15, 2026

(54) MASCARA COMPOSITION

(71) Applicant: Coty Inc., New York, NY (US)

(72) Inventors: Tatyana Zamyatin, New York, NY (US); Aleksandr Plyamovaty, New York, NY (US); Sepideh O Karkouti, Morris Plains, NJ (US); Marissa Solomon, Morris Plains, NJ (US)

(21) Appl. No.: 19/680,736

(22) Filed: May 18, 2026

Related U.S. Application Data
(63) Continuation of application No. PCT/US2024/059616, filed on Dec. 11, 2024.
(60) Provisional application No. 63/608,704, filed on Dec. 11, 2023.

(51) Int. Cl. A61K 8/06; A61K 8/25; A61K 8/36; A61K 8/37; A61K 8/73; A61K 8/81; A61K 8/92; A61K 8/96; A61Q 1/10

(52) U.S. Cl. CPC A61K 8/062; A61K 8/25; A61K 8/361; A61K 8/37; A61K 8/731; A61K 8/8147; A61K 8/8182; A61K 8/922; A61K 8/965; A61Q 1/10; A61K 2800/412; A61K 2800/48

(57) ABSTRACT

Various embodiments disclosed relate to a mascara composition. A composition may include an emulsifier having lamellar characteristics. A composition may include a water phase comprising: a thickener; and an absorbent powder, an oil phase comprising: a wax system including a first wax with a melting point above 80 degrees Celsius and a second wax with a melting point of 40 to 65 degrees Celsius; and a film former.

### CROSS-REFERENCE TO RELATED APPLICATION

This application is a continuation of PCT Application Serial No. PCT/US2024/059616, filed Dec. 11, 2024, which application claims priority to U.S. Provisional Patent Application Ser. No. 63/608,704 filed on Dec. 11, 2023, which applications and publications are incorporated herein by reference in their entirety.

### BACKGROUND

Mascara compositions have been used for many years to increase the thickness, length, and overall aesthetic appearance of human eyelashes. Mascara is a leading cosmetic product in today's consumer market, with reported global sales of over USD 6.95 billion in 2021. Mascara is a popular way for a consumer to adjust and enhance the look of their eyelashes. Mascara products can help build volume of eyelashes and darken the color of eyelashes. Different mascara formulations can be worn for various periods of time, and may vary in the application and removal process. Mascara can be applied with a variety of types of mascara brushes and other applicators that provide different benefits.

### SUMMARY OF THE DISCLOSURE

In some aspects, the techniques described herein relate to a mascara formulation including: an emulsifier having lamellar characteristics; a water phase including: a thickener; an absorbent powder; an oil phase including: a wax system including a first wax with a melting point above 80 degrees Celsius and a second wax with a melting point of 40 to 65 degrees Celsius; and a film former.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIGS. 1A-1B depict results of the Crockmeter testing of samples A to D in an example.

FIGS. 2A-2F depict photos of application of mascara samples in an example.

FIGS. 3A-3C depict photos of application of mascara samples in an example.

FIGS. 4A-4C depict photos of application of mascara samples in an example.

FIGS. 5A-5B depict photos of mascara color in an example.

FIGS. 6A-6C depict mascara brush testing in an example.

### DETAILED DESCRIPTION

Reference will now be made in detail to certain embodiments of the disclosed subject matter, examples of which are illustrated in part in the accompanying drawings. While the disclosed subject matter will be described in conjunction with the enumerated claims, it will be understood that the exemplified subject matter is not intended to limit the claims to the disclosed subject matter.

Discussed herein is a mascara formulation that allows for extreme wear with bold volume and easy removal. The formulation is water resistant and smudge proof but allows for easier removal. The formulation is defined, buildable volume with an elevated length. When the mascara formulation is applied, the lashes are coated with mascara. The film dries, leaving the lash film plasticized and protected from external conditions. Later, the mascara is easily removed as a tubing form.

The formulation includes a unique film forming system with acrylates copolymer. The formulation additionally includes a wax system with absorbent cotton powder. The water phase of the formulation includes several thickeners, including water soluble clay and hydroxyethylcellulose. The emulsifier is an emulsifier with lamellar properties, containing sunflower phospholipids.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section.

In the methods described herein, the acts can be carried out in any order without departing from the principles of the invention, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

In the event of inconsistent usages between this document and any documents so incorporated by reference, the usage in this document controls.

The term “about” as used herein can allow for a degree of variability in a value or range, for example, within 10%, within 5%, or within 1% of a stated value or of a stated limit of a range, and includes the exact stated value or range.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%.

The term “substantially free of” as used herein can mean having none or having a trivial amount of, such that the amount of material present does not affect the material properties of the composition including the material, such that about 0 wt % to about 5 wt % of the composition is the material, or about 0 wt % to about 1 wt %, or about 5 wt % or less, or less than or equal to about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less, or about 0 wt %.

As used in this application, the term “sufficient wear” refers to the ability of a mascara formulation to adhere to a user's eyelashes for a period of time comparable to conventional mascara formulations as assessed by Crockmeter testing under a modified AATC method.

### Mascara Composition

According to various aspects of the present disclosure, a mascara composition can deliver desired volume, wear, and removal. The mascara composition can have an instant volume. The mascara compositions can have good viscosity, wax components, powder, and polymer selection which contribute to the beneficial volume.

Discussed herein, the mascara composition is an oil in water emulsion with both a water phase and an oil phase, in addition to an emulsifier. The water phase of the formulation can include, for example, thickeners or gellants, film formers, and various story ingredients. The oil phase can include, for example, thickeners or gellants, film formers, various story ingredients, and a wax system, or combinations thereof. These various components of the mascara composition can include polymers, silicones, waxes, pigments, other components, and combinations thereof. The polymers can provide a flexible non-flaking film that also prevents the mascara composition from smudging. The silicones can improve water resistance and make the mascara wear longer. The waxes can provide adhesion and have an impact on thickening lashes.

The mascara composition can be considered to be a vegan formulation. In some examples 100 wt % of the components of the mascara composition are vegan. The term “vegan formulation” as used herein refers to a mascara composition that includes component(s) that are not derived from an animal product and to the extent possible not tested on an animal. The mascara composition can be made of “clean” or “green” components.

An example mascara formulation is summarized below in Table 1.

TABLE 1
Example mascara formulation.

| Component | Examples |
|---|---|
| Emulsifier | glyceryl Stearate citrate |
| | andpolyglyceryl-3 stearate |
| | stearic acid and cetyl alochol |
| Thickener | hydroxyethylcellulose |
| | water-dispersible clay |
| Waxes (melting point above 80 °C) | carnauba wax |
| Wax (melting point between 40 °C | berry wax, tribehenin |
| and 65 °C) | |
| Powder | cotton powder |
| Film forming system | Acrylate copolymer |
| | VP/Eicosene copolymer |
| Oils | Castor oil, argan oil |
| Emollient | C10-18 triglycerides |

The formulation in Table 1 is an example formulation, not an exhaustive list. Alternative or additional components can be included in the formulation.

Emulsifier. The mascara composition can include an emulsifier to aid in mixing of the water phase and the oil phase. The emulsifier can have both micellar and lamellar properties. In an example, the emulsifier can be Glyceryl Stearate Citrate, Polyglyceryl-3 Stearate, and Hydrogenated Lecithin, Steric Acid, and combinations thereof. In an example, the emulsifier can be about 2.0 to about 4.0 wt % of the formulation, or about 2.0, 2.5, 3.0, 3.5, or 4.0 wt % of the formulation.

Emollient. The mascara composition can include one or more emollients or emulsion stabilizers. For example, the emollient can be C10-18 triglycerides. In an example, the emollient can be about 0.00 to 0.40 wt % of the formulation, or about 0.0, 0.05, 0.10, 0.15, 0.20, 0.25, 0.30, 0.35, or 0.40 wt % of the formulation. In some cases, the formulation can additionally include an emulsion stabilizer, such as hydroxyethylcellulose. Such an emulsion stabilizer can be present in in an amount of about 0.00 to 0.50 wt % of the formulation, or about 0.0, 0.1, 0.2, 0.3, or 0.5 wt % of the formulation.

Thickener or Gellant. The mascara composition can include one or more thickeners or gellants in the water phase. The thickeners or gellants can aid in the viscosity and tack of the mascara formulation, and aid in bold volume. The thickeners or gellants can include, for example, clay, gum, or cetyl alcohol.

The thickener can include, for example, hydroxyethylcellulose. The hydroxyethylcellulose can be provided in an amount of about 0.1 to about 0.5 wt % of the formulation, or about 0.1, 0.2, 0.3, 0.4, or 0.5 wt % of the formulation.

The thickener can include, for example, a water-dispersible clay, such as a hectorite clay. Such a water soluble clay can be provided in an amount of about 0.1 to about 0.7% of the formulation, or about 0.1, 0.2, 0.3, 0.4, 0.5, 0.6, or 0.7 wt % of the formulation.

Waxes. The mascara composition can include one or more waxes in the water phase. The waxes can help provide for removal of the mascara composition. The waxes can include, for example, a first wax having a melting point of above about 80° C., and a second wax having a melting point of about 40° C. to about 65° C. The first wax and the second wax can be provided at a ratio of about 1:2 to about 1:3.

In an example, the first wax can be either an organic or a synthetic wax. In an example, the first wax can be carnauba wax. In an example, the first wax can have a crystalline structure. The first wax can have a melting point of above about 80° C.

In an example, the second wax can be an either an organic or a synthetic wax. In an example, the second wax can be berry wax or tribehenin. In an example, the second wax can have an amorphous structure. The second wax can have a melting point of about 40° C. to about 65° C.

The wax system can be in an amount of about 5 wt % to about 10 wt % of the formulation, or about 5, 5.5, 6, 6.5, 7, 7.5, 8, 8.5, 9, 9.5, or about 10 wt % of the formulation. The first wax can be in an amount of about 1 wt % to about 5 wt % of the formulation, or about 1, 1.5, 2, 2.5, 3, 3.5, 4, 4.5, or 5 wt % of the formulation. The second wax can be in an amount of about 3 wt % to about 7 wt % of the formulation, or about 3, 3.5, 4, 4.5, 5, 5.5, 6, 6.5, or 7 wt % of the formulation.

Powder. The mascara composition can include an absorbent powder to aid in volume. The absorbent powder can be, for example, cotton powder. The powder can be a bulking agent. In an example, fiber can be used.

The absorbent powder can have a mean particle size of about 15 to 35 μm, or of about 15, 20, 25, 30, or 35 μm in an example. The absorbent powder can be provided in an amount of about 0.1 wt % to 2 wt % of the formulation, or of about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, or 2.0 wt % of the formulation.

Film Former. The mascara composition can include one or more film formers in the water and/or oil phases. The film formers can aid in the formation of a flexible and non-flaking film, and help prevent smudging while increasing wear of the mascara formulation. The film formers can include, for example, polymers. The film formers can include, for example, acrylate copolymer and VP/Eicosene copolymer. The first film former and the second film former can be provided at a ratio of about 4:1 to about 8:1.

In an example, the acrylate copolymer can be provided in an amount of about 5 to about 20 wt % of the formulation, for example 5, 10, 15, or 20 wt %. In additional examples, the film formers can include acrylates copolymer aqua, or acrylates copolymer.

In an example, the VP/Eicosene copolymer can be in the oil phase. The VP/Eicosene can be provided in an amount of about 2.5 to about 3.0 wt % of the formulation, for example 2.5, 2.6, 2.7, 2.8, 2.9, or 3.0 wt % of the formulation. In additional examples, the film formers can include Trimethylsiloxysilicate and Polypropylsilsesquioxane.

Rheology modifying components. The mascara formulation can further include a rheology modifying component. The rheology modifying component can be in a range of from about 2 wt % to about 15 wt % of the mascara formulation, about 8 wt % to about 12 wt % of the mascara formulation, less than, equal to, or greater than about 2 wt %, 3, 4, 5, 6, 7, 8, 9, 10, 11, 12, 13, 14, or about 15 wt % of the mascara formulation. Examples of suitable rheology modifying components include thickeners.

Thickeners for use in the present mascara formulations can be selected from the group consisting of waxes such as carnauba wax, candelilla wax, and polyethylene wax; particles such as disteardimonium hectorite, kaolin, silica, and magnesium carbonate; polymers; viscous hydrocarbons; and combinations thereof. Waxes are selected to maintain the film durability of the mascara formulation. In another example, hydroxyethylcellulose can be used as a thickener, such as in an amount of about 0.0 to about 0.8 wt % of the formulation. In another example, clay can be used as a thickener such as in an amount of about 0.0 to about 0.8 wt % of the formulation. The thickener(s) can contribute both to thickness and stability.

Story Ingredients. A variety of story ingredients can be included in the mascara composition, including but not limited to conditioning agents, fibers, butter, functional ingredients, fragrance, and others. In an example, the mascara composition can include pH modifying ingredients, such as arginine.

Pigments. The mascara composition can include one or more pigments. The pigments, such as black, brown, or dark blue pigments, can be used for coloration. In some cases pearls or glitters can be used. The instantly disclosed mascara formulation can include a colorant or pigment component, which can be present in the mascara formulation in a range of from about 5 wt % to about 20 wt % of the mascara formulation, about 8 wt % to about 12 wt % of the mascara formulation, less than, equal to, or greater than about 5 wt %, 10, 15, or 20 wt % of the mascara formulation. Colorants suitable for use in the present mascara formulations include, but are not limited to, dyes, pigments, lakes, and mixture thereof. (e.g., organic or inorganic pigments and colorants approved for use in eye-area cosmetics by CTFA and/or the FDA.) Exemplary inorganic pigments include particles of iron oxides (e.g., yellow, brown, red, black), titanium dioxides, iron sulfides, ultramarines, chromium oxides (e.g., green) or other conventional pigments used in cosmetic formulations. Examples of organic pigments include D&C Black No. 2, D&C Black No. 3, FD&C Red No. 40, D&C Green No. 5, FD&C Blue No. 1, and FD&C Yellow No. 5. Examples of lake dyes include various acid dyes which are laked with aluminum, calcium or barium. Additional colorants for use herein include annatto, caramel, carmine, β-carotene, bismuth oxychloride, ferric ammonium ferrocyanide, ferric ferrocyanide, chromium hydroxides (e.g., green), guanine, mica, aluminum powder, bronze powder, copper powder, manganese violet, zinc oxide. Suitable colorants along with their chemical structure are described in, e.g., 21 C.F.R. Part 74 and in the CTFA Cosmetic Ingredient Handbook, (1988), published by the Cosmetics, Toiletry and Fragrances Association, Inc. Other colorants may also be used as they are developed and determined safe.

Other additives. The mascara formulation can further include an oil-soluble or dispersible additive which may include fatty alcohols such as Guerbet alcohols based on fatty alcohols having from 6 to 30, preferably from 10 to 20 carbon atoms including lauryl alcohol, cetyl alcohol, stearyl alcohol, cetearyl alcohol, oleyl alcohol, benzoates of C₁₂-C₁₅ alcohols, acetylated lanolin alcohol, etc. Especially suitable is stearyl alcohol. The oil-soluble or dispersible additive may include fatty acids such as Linear fatty acids of C₆-C₂₄, branched C₆-C₁₃carboxylic acids, hydroxycarboxylic acids, caproic acid, caprylic acid, 2-ethylhexanoic acid, capric acid, lauric acid, isotridecanoic acid, myristic acid, palmitic acid, palmitoleic acid, stearic acid, isostearic acid, oleic acid, elaidic acid, petroselinic acid, linoleic acid, linolenic acid, elaeostearic acid, arachidic acid, gadoleic acid, behenic acid and erucic acid and technical-grade mixtures thereof (obtained, for example, in the pressure removal of natural fats and oils, in the reduction of aldehydes from Roelen's oxosynthesis or in the dimerization of unsaturated fatty acids). Further components that can be used are dicarboxylic acids of C₂-C₁₂, such as adipic acid, succinic acid, and maleic acid. Aromatic carboxylic acids, saturated and/or unsaturated, especially benzoic acid, can be used. Additional components that can be used as the oil soluble or dispersible additive include carboxylic acid salts; alkaline soaps of sodium, potassium and ammonium; metallic soaps of calcium or magnesium; organic basis soaps such as lauric, palmitic, stearic and oleic acid, etc., alkyl phosphates or phosphoric acid esters: acid phosphate, diethanolamine phosphate, potassium cetyl phosphate.

Other useful oil-soluble or dispersible additives comprise mild surfactants, super-fatting agents, consistency regulators, additional thickeners, polymers, stabilizers, biologically active ingredients, deodorizing active ingredients, anti-dandruff agents, film formers, swelling agents, UV light-protective factors, antioxidants, preservatives such as caprylyl glycol, insect repellents, solubilizers, colorants, bacteria-inhibiting agents, hair conditioning agents, vitamins, and the like.

Other components. There are any number of further components that can be included in the mascara formulation such as a surfactant component, a skin conditioner component such as arginine, glycerin, 2,3-butanediol, or propanediol, a humectant component, or another additive component. Other additives can include chamomile and cucumber extract, jojoba oil, camellia oil, aloe butter, maracuja oil, cell-u-lash, marula oil, castor oil, ceramide 2, avocado butter, or a mixture thereof. In additional examples, oils such as castor oil and argan oil can be used. In some cases, antioxidants such as vitamin E can be used. In some cases, hair conditioning agents such as panthenol can be used.

### Method of Making Mascara Formulation

The instantly described mascara formulations can be made according to a variety of suitable methods. As an example, water and the ingredients in the water phase can be added to a beaker and exposed to low shear conditions (about 100 rpm) at room temperatures (about 25° C.). All waxes can be melted (e.g., change from a solid state to a liquid or molten state). Once the waxes are sufficiently melted, other components such as pigments, emulsifiers, and the like are added.

### Method of Using

The mascara formulation can be contained in an applicator assembly. A variety of conventional applicators are known in the art (e.g., molded plastic applicators and twisted-wire brush applicators).

Mascara formulations may act as a paste or semi-solid on the brush, rather than as a flowable liquid. Thus, the primary method of depositing such mascaras onto the mascara brush is by inserting the brush into a supply of mascara, typically contained in a bottle or similar container, so that the mascara is deposited on the head (i.e., bristles and core) of the brush. Excess mascara is removed from the head of the brush, especially the outermost portions of the bristles spaced away from the core, via a wiper when the brush is withdrawn from the mascara container. Ideally, a suitable amount of mascara remains at least on the core of the brush for application to the eyelashes of a user.

When the brush is used as intended, the lashes of a user are moved past the bristles of the brush via the spaces between the bristles such that the lashes come into contact with the mascara-containing core and, optionally, portions of the bristles (e.g., the inner portions). Thus, it is the characteristics of the mascara (e.g., coherent strength & rigidity) in combination with the applicator and wiper that provides the primary mechanism of product control and dosing. Because conventional mascaras tend to be thick enough for the mascara to remain on the core of the brush, it is not uncommon for the spacing of the bristles to be determined primarily by the desired lash separation/clustering. But with the desire to provide lower viscosity mascaras, the spacing of the bristles becomes important for providing suitable product retention on the brush as well as the desired amount of lash separation/clustering.

A suitable mascara product according to the present disclosure includes a container (e.g., bottle or the like) for storing a supply of the present semi-permanent mascara formulation; an applicator (e.g., twisted-wire brush or molded plastic brush) for transferring mascara from the container to the eyelashes of a user; and a supply of the present semi-permanent mascara formulation disposed in the container. The applicator may include a handle which is removably and/or re attachably joined to the container (e.g., with screw threads, snap collar, or the like).

### EXAMPLES

### Example 1. Mascara Formulation

For each of the Examples below, a sample mascara composition was produced with the following components, except where otherwise noted:

TABLE 3
Mascara Composition.

| Component | Examples | Source | Amount |
|---|---|---|---|
| Emulsifier | Glyceryl Stearate | Lucase Meyer | 0.0 to 9.0% |
| | | Cosmetics | |
| Emulsifier | Stearic Acid | Lucase Meyer | 0.0 to 5.0% |
| | | Cosmetics | |
| Thickener | Natrosol ® | Ashland | 0.0 to 0.7% |
| Thickener | water-dispersible | ELEMENTIS | 0.0 to 0.7% |
| | clay | | |
| Thickener | hectorite clay | ELEMENTIS | 0.0 to 0.5% |
| Wax | carnauba wax | KAHL GMBH & CO | 1.0 to 4.0% |
| | | KG | |
| Wax | tribehenin | CRODA | 2.0 to 7.0% |
| Powder | cotton powder | ACTIVE CONCEPTS | 0.01 to 2% |
| Film former | Unimer ® | Givaudan Active | 1.0 to 5.0% |
| | | Beauty | |
| Film former | Acrylate | Worlée | 0 to 20% |
| | copolymer | | |

These components were provided in the same types and amounts for each of the samples discussed and tested in Examples 2 to 4 below except where otherwise noted.

### Example 2. Film Forming System

Samples of the film forming system were made and tested. The first polymer, an acrylate copolymer (Ammonium Acrylates Copolymer), and the second polymer, a VP/Eicosene copolymer (Unimer U-15) were provided in the following ratios:

TABLE 3
Sample film forming systems.

| Sample | Acrylate copolymer | VP/Eicosene copolymer | Ratio |
|---|---|---|---|
| A | 30% | 0% | 30:0 |
| B | 20% | 10% | 2:1 |
| C | 10% | 20% | 1:2 |
| D | 0% | 30% | 30:0 |

These Samples A to D were tested for wear (or “colorfastness”) in a rub test with a Crockmeter under a modified AATCC method 116-2013-Colorfastness to Crocking. Several different rubbing insult types are tested: rub testing as a dry rub, with water, saline, and artificial sebum (squalene). Each of these liquids represents a different liquid that a user might come into contact with throughout the day while wear the mascara composition. Each liquid (water, saline, squalene) could potentially smudge, flake, or otherwise move the formulation from the user's lashes. Moreover, some of these liquids, such as water, could potentially be used at the end of the day to remove the formulation from the user's lashes. Preferably, the formulation does not smudge or smear under the effects of these liquids, but is also easily removable at the end of the say, such as with the application of water by the user.

For the rub testing, each of the samples was prepared on a cotton linen sheets. One each of the dry, water, saline, and squalene slides were produced with each of Sample A to D. For the wet samples, a small 20 μL drop of the liquid (water, saline, or squalene) was pipetted onto the sample. The sheets were then put into the Crockmeter according to the AATCC method. A manual crank of 10 was used. The rub testing of the sheets on the Crockmeter was iterated.

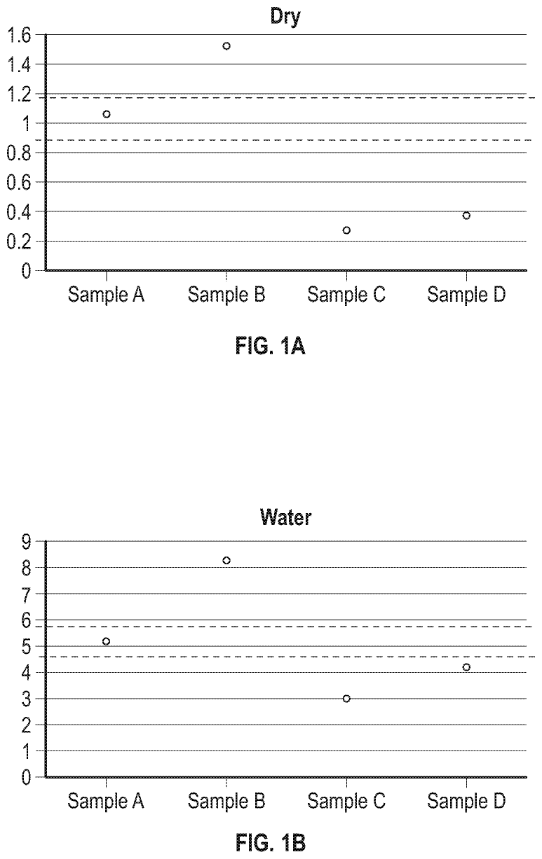

The results of the Crockmeter testing of samples A to D are depicted in FIGS. 1A to 1B. A very low Crockmeter value can indicate the sample was difficult to remove, while a very high Crockmeter value can indicate the sample was prone to smudging.

Here, the Crockmeter testing of the dry samples and the water samples are summarized. For the dry samples in FIG. 1A, the Crockmeter rating is shown. For the dry Crockmeter testing, good sample would preferably have a rating of between about 0.8 and 1.2. The lower number of the Sample C and Sample D formed too strong a film, which was not easily removable.

In FIG. 1B, the Crockmeter rating is shown for the water samples. For the wet samples in FIG. 1B, a preferred Crockmeter reading would be between about 4.5 and 6.0. Here, Sample C, with an Acrylate to VP/Eicosene ratio of 1:2, and a Crockmeter rating of 3, was difficult to remove. By comparison, the Sample B with an Acrylate to VP/Eicosene ratio of 2:1 and a Crockmeter rating of above 8, was subject to smudging.

Additional film forming system samples were provided in the following ratios:

TABLE 4
Sample film forming systems.

| Sample | Acrylate copolymer | VP/Eicosene copolymer | Ratio |
|---|---|---|---|
| E | 12% | 3% | 4:1 |
| F | 13.5% | 1.5% | 9:1 |
| G | 10% | 5% | 2:1 |

These film forming system samples were tested with the Crockmeter in the same way as described above with reference to Samples A to D. The resulting Crockmeter scores for these Samples tested with dry, squalene, water, and saline methods, are summarized below in Table 4:

TABLE 5
Sample film former ratios.

| Sample | DRY | SQUALANE | WATER | SALINE |
|---|---|---|---|---|
| E | 1.06 | 4.02 | 5.17 | 6.22 |
| F | 1.14 | 3.77 | 3.25 | 2.83 |
| G | 1.33 | 5.22 | 3.29 | 4.05 |

Overall, the Sample E, which had an Acrylate to VP/Eicosene ratio of 4:1, performed best across the Crockmeter testing.

The Sample E had a good result for testing with water of 5.17, indicating that the Sample E could be easily removed, but the number was not low enough to allow for extensive smudging or flaking of the formulation when water was applied. By comparison, the Samples F and G both had low Crockmeter values, from about 3.25 to 3.29, when tested with water. These values indicated that Sample F and Sample G would have been difficult to remove with just water.

Sample E also had good results for the squalene (representing sebum that naturally builds on a user) and saline (representing sweat), indicating that the formulation could avoid smudging. By comparison, Sample F and Sample G were less washable. The Sample G produced a harder dry film compared to Sample E.

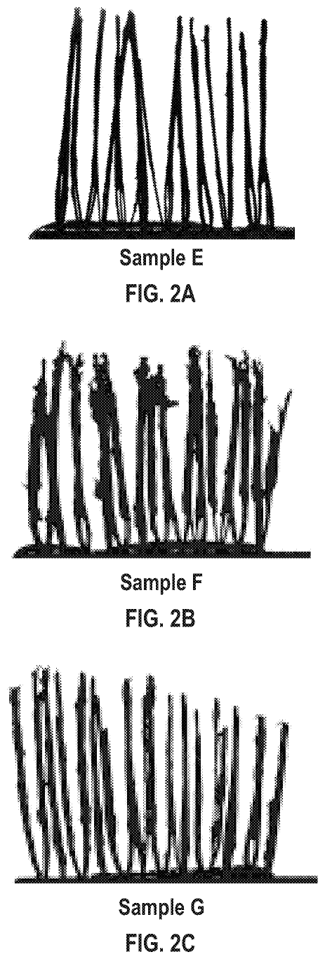

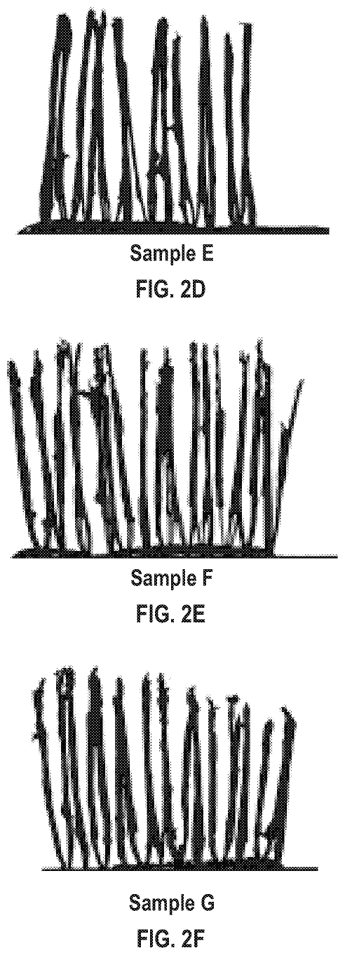

The Samples E, F, and G were also tested for buildable volume. This test was conducted by using a standard mascara brush and applying the sample formulations to synthetic lashes in multiple strokes. Photographs of the resulting applications are shown in FIGS. 2A to 2F. Here, FIGS. 2A to 2C depict the application of 10 strokes of the formulation. FIGS. 2D to 2F depict the application of 20 strokes of the formulation.

Here, Sample E (in FIGS. 2A and 2D) visually shows cleaner lashes compared to Samples F and G (in FIGS. 2B, 2C, 2E, 2F). There is visual clumping between lashes in Sample F and Sample G that is not present with Sample E. In particular, Sample F appears to have lashes stuck together. Overall, Sample E provided good buildable volume.

### Example 3. Powder

Sample mascara formulations using different powders were tested to observe the effect on volume of the formulation. The following Samples were prepared:

TABLE 6
Sample powder formulations.

| Sample | Powder |
|---|---|
| H | 1% cotton powder |
| I | 1% silica |

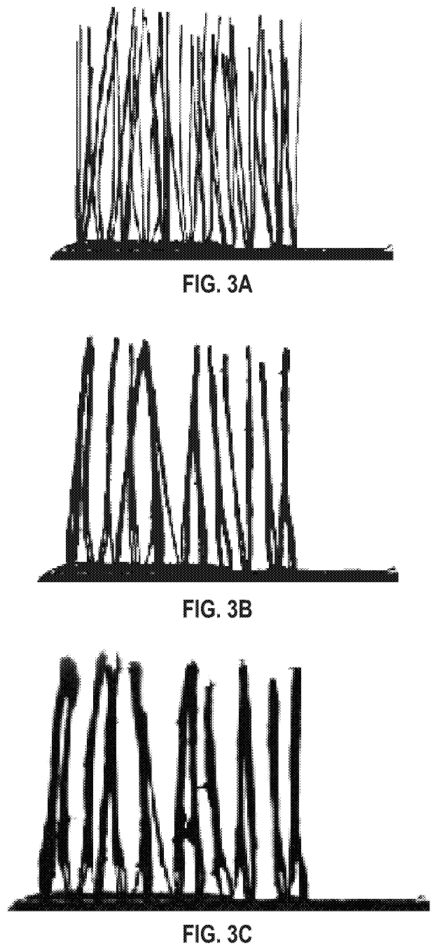

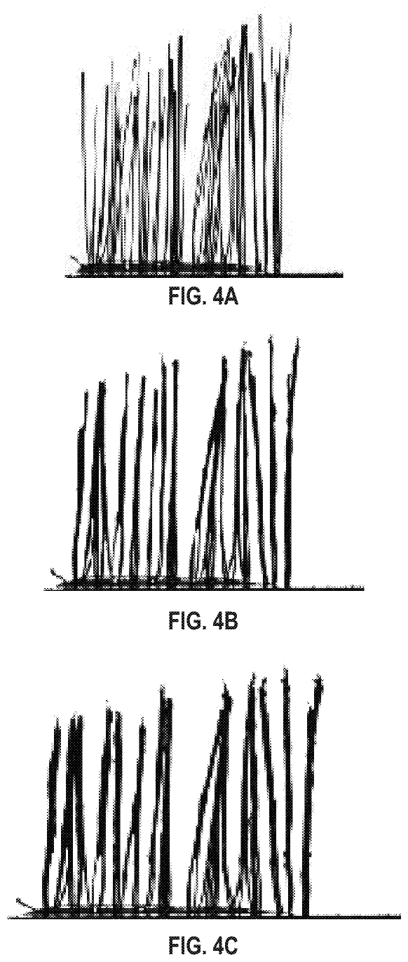

Both Sample H and Sample 1 were tested for buildable volume in a similar fashion to that described above in Example 2. This test was conducted by using a standard mascara brush and applying the sample formulations to synthetic lashes in multiple stokes. Photographs of the resulting applications are shown in FIGS. 3A to 4C. FIGS. 3A to 3C depict Sample H, including 1% cotton powder, while FIGS. 4A to 4C depict Sample I, including 1% silica for comparison.

FIG. 3A depicts a bare lash without application of the formulation. FIG. 3B depicts a 10 stroke application of the Sample H with 1% cotton powder. FIG. 3C depicts a 20 stroke application of the Sample H with 1% cotton powder.

FIG. 4A depicts a bare lash without application of the formulation. FIG. 4B depicts a 10 stroke application of the Sample 1 with 1% silica. FIG. 4C depicts a 20 stroke application of the Sample 1 with 1% silica.

As can be seen in the photographs, the Sample H with the 1% cotton powder provided bigger and more visually present volume compared to the Sample I with 1% silica.

### Example 4. Emulsifier

Sample mascara formulations using different emulsifiers were tested to observe the effect on the formulation. The following Samples were prepared:

TABLE 7
Sample emulsifier formulations.

| Sample | Emulsifier |
|---|---|
| J | Heliofeel ® |
| K | Amisol ® |

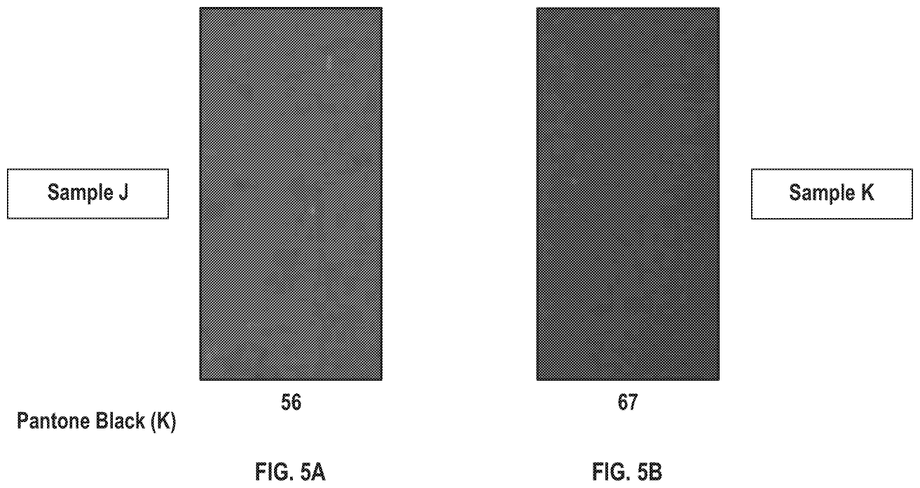

The Samples J and K were tested for color, using a blackness evaluation protocol. Here, a drawdown of the samples was made on a glossy card (Lanetta), using a 6 mL bird applicator. The drawdown was dried for 30 minutes. A picture was recorded of the drawdown in a light box using a camera. The PANTONE color was defined of each photo using Photoshop by cyan (C), magenta (M), yellow (Y), and black (K) numerically. The results photographs were compared to the PANTONE black (K) numerically. The resulting photos are in FIG. 5A (showing Sample J) and FIG. 5B (showing Sample K). On review, the Sample K had a 67 rating compared to the PANTONE black, while the Sample J had a 56 rating. Overall, the use of the Heliofeel® intensified the black color of the formulation.

### Example 5. Brush Testing

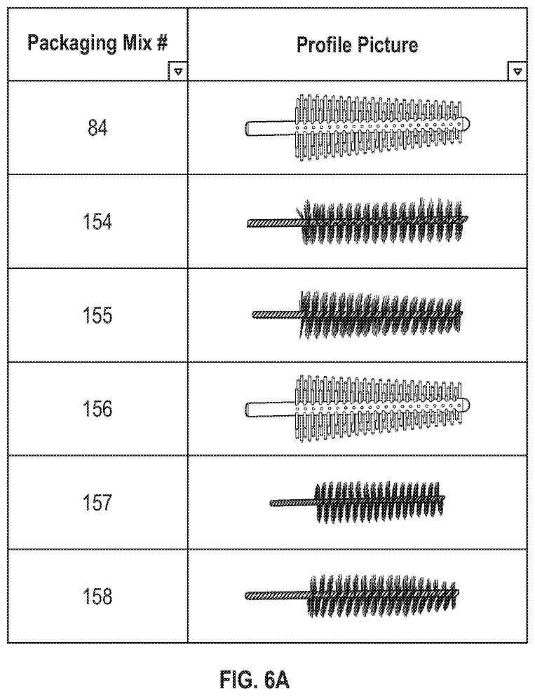

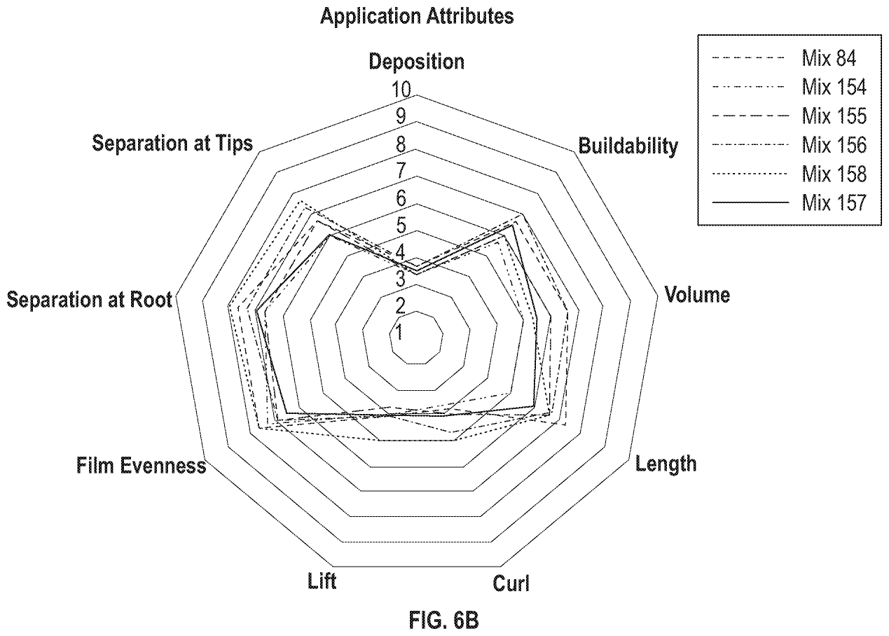

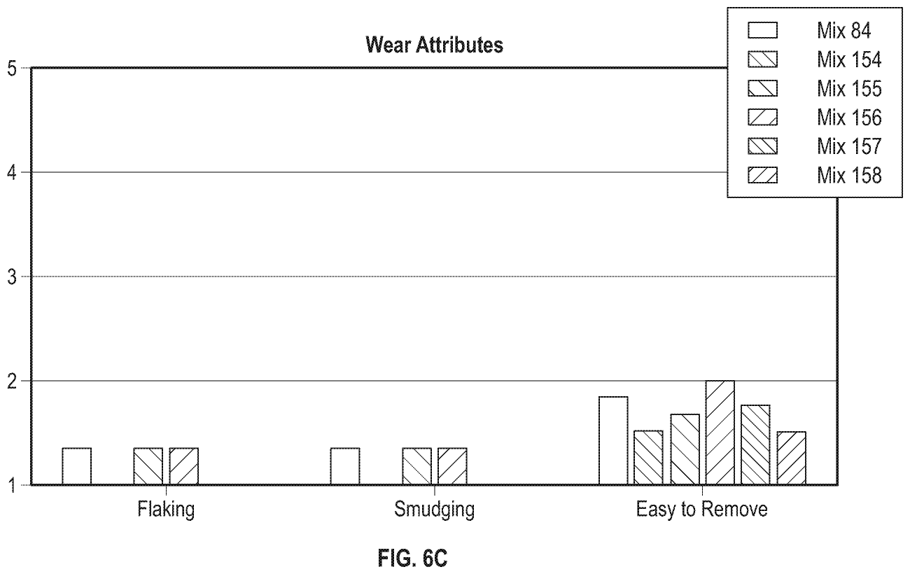

Sample mascara formulations using different mascara brushes were tested to observe the effect with the formulation. The different brushes tested are shown in FIG. 6A. The resulting application attributes are shown and evaluated in FIGS. 6B and 6C. Overall, the formulations 154 and 156 with the respective brushed performed the best.

### Various Notes & Examples

In some aspects, the techniques described herein relate to a mascara formulation including: an emulsifier having lamellar characteristics; a water phase including: a thickener; and an absorbent powder; an oil phase including: a wax system including a first wax with a melting point above 80 degrees Celsius and a second wax with a melting point of 40 to 65 degrees Celsius; and a film former.

In some aspects, the techniques described herein relate to a mascara formulation, wherein the first wax and the second wax are provided at a ratio of between 1:2 to 1:3.

In some aspects, the techniques described herein relate to a mascara formulation, wherein the emulsifier includes glycerol stearate, steric acid, or combinations thereof.

In some aspects, the techniques described herein relate to a mascara formulation, wherein the emulsifier includes 2.0% to 4.0% of the formulation.

In some aspects, the techniques described herein relate to a mascara formulation, wherein the thickener includes hydroxyethylcellulose.

In some aspects, the techniques described herein relate to a mascara formulation, wherein hydroxyethylcellulose includes 0.1% to 0.5% of the formulation.

In some aspects, the techniques described herein relate to a mascara formulation, wherein the thickener includes water dispersible clay.

In some aspects, the techniques described herein relate to a mascara formulation, wherein the water dispersible clay includes 0.1% to 0.7% of the formulation.

In some aspects, the techniques described herein relate to a mascara formulation, wherein the wax includes carnauba wax, tribehenin, or a combination thereof.

In some aspects, the techniques described herein relate to a mascara formulation, wherein the absorbent powder has a mean particle size of 15 μm to 35 μm.

In some aspects, the techniques described herein relate to a mascara formulation, wherein the absorbent powder includes cotton powder.

In some aspects, the techniques described herein relate to a mascara formulation, wherein the film former includes VP/Eicosene copolymer.

In some aspects, the techniques described herein relate to a mascara formulation, wherein the film former is 2.5% to 3.5% of the formulation.

In some aspects, the techniques described herein relate to a mascara formulation, further including a second film former in the water phase.

In some aspects, the techniques described herein relate to a mascara formulation, wherein the second film former includes an aluminum acrylate copolymer.

In some aspects, the techniques described herein relate to a mascara formulation, wherein the second film former is 5% to 20% of the formulation.

In some aspects, the techniques described herein relate to a mascara formulation, wherein the first film former and the second film former are provided at a ratio of 1:8.

In some aspects, the techniques described herein relate to a mascara formulation, further including an emollient.

In some aspects, the techniques described herein relate to a mascara formulation, wherein the first wax, the second wax, and the emollient have a ratio of about 1:1:0.07.

In some aspects, the techniques described herein relate to a mascara formulation, wherein the emollient includes C10-18 triglycerides.

Each of these non-limiting examples can stand on its own or can be combined in various permutations or combinations with one or more of the other examples.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

What is claimed is:

1. A mascara formulation comprising:
an emulsifier having lamellar characteristics;
a water phase comprising:
a thickener;
an absorbent powder;
an oil phase comprising;
a wax system including a first wax with a melting point above 80 degrees Celsius and a second wax with a melting point of 40 to 65 degrees Celsius; and
a film former.

2. The mascara formulation of claim 1, wherein the first wax and the second wax are provided at a ratio of between 1:2 to 1:3.

3. The mascara formulation of claim 1, wherein the emulsifier comprises glycerol stearate, steric acid, or combinations thereof.

4. The mascara formulation of claim 1, wherein the emulsifier comprises 2.0% to 4.0% of the formulation.

5. The mascara formulation of claim 1, wherein the thickener comprises hydroxyethylcellulose.

6. The mascara formulation of claim 5, wherein hydroxyethylcellulose comprises 0.1% to 0.5% of the formulation.

7. The mascara formulation of claim 1, wherein the thickener comprises water dispersible clay.

8. The mascara formulation of claim 7, wherein the water dispersible clay comprises 0.1% to 0.7% of the formulation.

9. The mascara formulation of claim 1, wherein the wax system comprises carnauba wax, tribehenin, or a combination thereof.

10. The mascara formulation of claim 1, wherein the absorbent powder has a mean particle size of 15 μm to 35 μm.

11. The mascara formulation of claim 1, wherein the absorbent powder comprises cotton powder.

12. The mascara formulation of claim 1, wherein the film former comprises VP/Eicosene copolymer.

13. The mascara formulation of claim 12, wherein the film former is 2.5% to 3.5% of the formulation.

14. The mascara formulation of claim 1, further comprising a second film former in the water phase.

15. The mascara formulation of claim 14, wherein the second film former comprises an aluminum acrylate copolymer.

16. The mascara formulation of claim 15, wherein the second film former is 5% to 20% of the formulation.

17. The mascara formulation of claim 14, wherein the film former and the second film former are provided at a ratio of 1:8.

18. The mascara formulation of claim 1, further comprising an emollient.

19. The mascara formulation of claim 18, wherein the first wax, the second wax, and the emollient have a ratio of about 1:1:0.07.

20. The mascara formulation of claim 18, wherein the emollient comprises C10-18 triglycerides.
